(19) United States
(12) Patent Application Publication
(10) Pub. No.: US 2002/0058010 A1
(43) Pub. Date: May 16, 2002

(54) Foaming cosmetic cream for treating greasy skin and methods for using the same

(75) Inventors: Elisabeth Picard-Lesboueyries, Velizy (FR); Veronique Guillou, Antony (FR)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: L'OREAL, Paris (FR)

(21) Appl. No.: 09/941,589

(22) Filed: Aug. 30, 2001

(30) Foreign Application Priority Data
Aug. 31, 2000 (FR) 0011130

(51) Int. Cl. A61K 7/00; A61K 9/00

(52) U.S. Cl. 424/43

(57) ABSTRACT

The present patent application relates to a foaming composition for topical application, containing (1) a surfactant system such that at least one paracrystalline phase of direct or cubic hexagonal type appears when the temperature increases above 30° C. and such that this paracrystalline phase remain present up to at least 45° C., and (2) an active agent chosen from antibiotics and anti-seborrhoeic agents. The surfactant system which allows such a paracrystalline phase to be obtained preferably comprises at least one water-soluble surfactant and at least one water-insoluble surfactant. It preferably comprises at least one water-soluble soap. These compositions exist in the form of creams with good physical stability at ambient temperature and even up to at least 45° C. They may be used n cosmetics or dermatology, for cleansing or treating greasy skin and/or acne-prone skin.

### CROSS REFERENCES TO RELATED APPLICATIONS

This application claims priority to French Patent Application No. 001 1130, filed on Aug. 31, 2001, and which is incorporated herein by reference in its entirety.

### BACKGROUND OF THE INVENTION

1. Field of the Invention

The present invention relates to rinsable foaming compositions constituting a cream for topical application, which contain a particular surfactant system and an antibiotic or anti-seborrhoeic active agent, and having good physical stability up to at least 45° C. The present invention also relates to the use of such compositions in cosmetics or dermatology, especially for cleansing or treating greasy skin and/or acne-prone skin.

2. Discussion of the Background

Cleansing the skin is very important for facial care. The cleansing should be as effective as possible since greasy residues such as excess sebum, residues of cosmetic products used daily and make-up products, in particular “waterproof” products, accumulate in the folds of the skin and can obstruct the pores of the skin and lead to the appearance of spots.

Greasy skin and/or acne-prone skin require suitable cleansing capable of removing the sebum entirely, purifying the surface of the skin and cleaning the pores deep down. Greasy or hyperseborrhoeic skin is characterized by a thick shiny skin, occasionally of oily appearance, with dilated pilosebaceous pores. In addition, it promotes desquamation. Sebaceous hypersecretion is usually associated with a hyperandrogeny due either to an overproduction of androgens by an endocrine gland or to a peripheral overproduction in the sebaceous gland from the surrounding androgens and/or proandrogens. The androgen which induces lipid synthesis in the sebocyte nucleus is dihydrotestosterone (DHT) which originates from the reduction of testosterone with 5-α-reductase. The skin has a granular general appearance and, depending on the case, may show well-known imperfections such as whiteheads, blackheads, small spots or sebaceous microcysts which are quite often preludes to the development of acne conditions.

Moreover, acne vulgaris, commonly known as acne, is a skin disorder which affects a large number of people. It develops especially in people with hyperseborrhoea. Acne arises when the sebaceous follicles, located on the face and the back, become obstructed with sebum and epithelial cells. The obstruction of the follicle creates a micro-comedone which may develop into a comedone or an inflammatory lesion. A resident anaerobic bacterium, Propionibacterium acnes, proliferates in this environment rich in sebum and follicular cells. This bacterium may produce an inflammation locally, by releasing lipases which lead to the formation of free fatty acids. These fatty acids are themselves comedone-generating and primary irritants. Specifically, chemotactic factors are secreted by P. acnes and act by attracting neutrophils from the follicular wall.

Active agents that are specific for greasy skin, such as, for example, salicylic acid, benzoyl peroxide or triclosan, are used in skin cleansing compositions to avoid the appearance of such phenomena. Several types of cleansing products are known for greasy skin, and in particular foaming detergent aqueous gels and lotions. These products have a cleansing action by means of surfactants which place in suspension the greasy residues and pigments of the make-up products. They are effective and pleasant to use due to the fact that they foam and are easy to remove. However, they are generally relatively fluid, and it is difficult to thicken them while at the same time retaining good foaming properties.

In order to obtain good foaming performance while at the same time having a thick composition, attempts have been made to prepare foaming creams. The expression “foaming creams” means herein opaque, viscous compositions often sold in tubes and generally consisting of an aqueous medium containing a mixture of surfactants such as fatty acid salts (soaps) or anionic, nonionic or amphoteric synthetic surfactants, and other additives such as, for example, polymers, polyols or fillers. These creams intended in particular for cleansing the skin develop a foam when they are mixed with water. They may be used in two ways:

the first use consists of spreading the cream between the hands, applying it to the face or the body and then massaging it into the skin in the presence of water to develop the foam directly on the face or the body;

the other possible use of this type of product consists of developing the foam in the palms of the hands and then applying it to the face or the body. In both cases, the foam is then rinsed off.

However, most of the foaming creams currently available on the market have the drawback of being unstable under hot conditions, that is to say above 40° C. This means that, if they are stored for a few days at this temperature, they show macroscopic demixing, resulting in a separation into at least two phases. The creams thus demixed at a temperature markedly above room temperature are found to be heterogeneous after returning to room temperature and thus unusable on account of the degradation of the texture and foaming properties. The term “room temperature” means herein a temperate temperature, i.e. about 20 to 25° C. Such instability is harmful to the storage of the cream itself, and also to the stability of the active agent(s) contained therein.

Specifically, it is essential for this type of product to be stable over a wide range of temperatures, since, in its lifetime, the product may be exposed to temperatures ranging from −20° C. to +45° C. minimum depending on the climatic, storage and/or transportation conditions. For example, a cream transported in a car which is liable to remain in the sun for a long time, i.e. at a temperature which may easily reach 50° C., needs to conserve its stability. These foaming creams also need to be able to be used in hot countries without posing any problem as regards their transportation or storage.

It is well known that it is possible to prevent this phase-separation of a foaming cream by increasing, by means of adding polymers or fillers, the consistency of the product subjected to temperatures from +40° C. to +45° C. However, in this case, the product becomes very rigid at temperate, ambient temperatures and no longer corresponds to the properties desired for use on the skin, and in particular it becomes difficult to mix it with water and make it form a foam.

Thus, there remains a need for a foaming cream for treating greasy skin or acne-prone skin, which is stable up to at least 45° C., has a cream appearance which is maintained at ambient temperature even after passing to a higher temperature, and which has good foaming properties.

### SUMMARY OF THE INVENTION

Accordingly, it is one object of the present invention to provide novel compositions which are useful for treating greasy skin or acne-prone skin.

It is another object of the present invention to provide novel compositions which are useful for treating greasy skin or acne-prone skin which are stable up to at least 45° C.

It is another object of the present invention to provide novel compositions which are useful for treating greasy skin or acne-prone skin which have good foaming properties.

It is another object of the present invention to provide novel methods for treating greasy skin and/or acne-prone skin by treating the skin with such a composition.

These and other objects, which will become apparent during the following detailed description, have been achieved by the inventors' surprising discovery that foaming compositions may be obtained which are in the form of a cream with good stability, even at temperatures of +40° C. to +45° C., by using a surfactant system such that at least one paracrystalline phase of direct or cubic hexagonal type appears when the said composition is heated to a temperature above 30° C. and such that this paracrystalline phase remains present up to at least 45° C.

The fact that one paracrystalline phase of direct hexagonal or cubic type appears when the composition is heated to a temperature of greater than 30° C. and such that this paracrystalline phase remains present up to at least 45° C., means that this phase is present at a temperature ranging from 30° C. to 45° C.

In order to obtain the required stability, it is preferable for the paracrystalline phase formed (or liquid crystal) to be of the direct hexagonal phase type. It is not necessary for this paracrystalline phase to be present at ambient temperature, but it must imperatively appear above a temperature of between 30° C. and 45° C.

Foaming creams, which do not have a phase organization as mentioned above, are generally not stable at 45° C. At this temperature, they undergo a macroscopic demixing between at least two phases and are then unsuitable for the desired use when they are again at ambient temperature.

### DETAILED DESCRIPTION OF THE PREFERRED EMBODIMENTS

Thus, in a first embodiment, the present invention provides novel foaming compositions which exist as a cream for topical application, comprising, in an aqueous medium:

(1) a surfactant system such that at least one paracrystalline phase of direct and/or cubic hexagonal type appears when the temperature increases above 30° C. and such that this paracrystalline phase remains present up to at least 45° C.; and

(2) an active agent chosen from anti-seborrhoeic active agents and antimicrobial agents.

The obtained composition constitutes a opaque cream which has very good cosmetic properties (softness, creaminess), gives good foam and has a good stability for a long time and at elevated temperatures.

The compositions obtained are particularly suitable for removing sebum and for cleansing the pores, which constitute the main problem areas of greasy skin as regards cleansing.

The paracrystalline phase(s) present above +30° C. may be of direct or cubic hexagonal type, or may be a mixture of these two phases or a mixture of one of these phases or of these two phases with a phase of lamellar type. The paracrystalline phase(s) preferably comprise(s) at least one direct hexagonal phase.

In the present patent application, the terms “lamellar phase,” “direct hexagonal phase,” and “cubic phase” have the meanings usually given to them by those skilled in the art.

Thus, the term “lamellar phase” (phase D according to Ekwall, see Advances in Liquid Crystals, vol. 1, page 1-143, Acad. Press, 1975, Ed. G. H. Brown, which is incorporated herein by reference), means a liquid crystal phase with plane symmetry, comprising several amphiphilic bilayers arranged in parallel and separated by a liquid medium which is generally water.

The term “direct hexagonal phase” (phase F according to Ekwall, see Advances in Liquid Crystals, vol. 1, page 1-143, Acad. Press, 1975, Ed. G. H. Brown, which is incorporated herein by reference) means a liquid crystal phase corresponding to a hexagonal arrangement of parallel cylinders consisting of an amphiphile and separated by a liquid medium which is generally water. In a direct hexagonal phase, the continuous medium is aqueous.

The term “cubic phase” means a phase organized in a bipolar manner into separate hydrophilic and lipophilic domains, in close contact and forming a thermodynamically stable three-dimensional network with cubic symmetry. Such an organization has been described in particular in La Recherche, vol. 23, pp. 306-315 (March 1992) and in Lipid Technology, vol. 2, no. 2, pp. 42-45 (April 1990), which are incorporated herein by reference. According to the arrangement of the hydrophilic and lipophilic domains, the cubic phase is said to be of normal or inverse type. The term “cubic phase” used according to the present invention obviously combines the various types of cubic phase.

A more precise description of these phases may be found in the Revue Francaise des Corps Gras, No. 2, pp. 87 to 111, February 1969 (Lachampt and Vila, “Textures des phases paracristallines” [Textures of paracrystalline phases]), which is incorporated herein by reference.

Various techniques may be used to identify the constituent phases of the cream, and in particular (1) X-ray diffraction measurements at small angles and at large angles, and (2) observation by polarized-light optical microscopy.

1. X-ray Diffraction Technique:

The technique of x-ray diffraction is known as being one of the most relevant for demonstrating the organization of paracrystalline phases, in particular within a sample. The x-ray diffraction measurements may be carried out using a CGR Sigma 2060 generator equipped with a Cu anticathode Inel tube and a linear focusing chamber mounted in symmetrical transmission. The samples are introduced at ambient temperature into a measuring cell closed off with Mylar or Capton windows and placed in a temperature-controlled sample holder.

The diffraction spectra obtained with a wavelength λ=1.54 Angstroms (Kα line of copper) are recorded using a phosphorescent screen scanned with a Molecular Dynamics Phosphorlmager PSI laser scanning module. The detector/sample distance is adjusted to 133 mm, which gives access to reticular distances of between about 3 and 110 Angstroms. The spectra are recorded at different set temperatures.

With this technique, the paracrystalline phases are characterized by the presence, at small diffraction angles, of a series of several fine lines due to Bragg reflections and corresponding to distances: d1, d2. . . dn with distance ratios d1/d1, d1/d2, . . . , d1/dn which are characteristic of phases of each type, as indicated, for example, in “La structure des colloides d'association, I. Les phases liquides cristallines des systems amphiphile-eau” [The structure of association colloids, I. The liquid crystal phases of amphiphile-water systems], V. Luzzati, H. Mustachi, A. Skoulios and F. Husson, Acta Cryst., vol. 13, pp. 660-667 (1960), or in J. M. Seddon, Biochimica and Biophysica Acta, vol. 1031, pp. 1-69 (1990), which are incorporated herein by reference. Thus, for a phase of lamellar structure and in particular for the paracrystalline phase of fluid lamellar type generally denoted by La and also known as a “neat phase,” the distance ratios are equal to: 1, 2, 3, 4, . . . . For the paracrystalline phase of the direct hexagonal type generally denoted by H1 or E and also known as the “middle phase,” the distance ratios are equal to: 1, √3, 2, √7, . . . . At large diffraction angles, the paracrystalline phases have a band centred on a distance of about 4.5 Angstroms, while the crystalline phases lead to fine lines.

2. Observations by Optical Microscopy:

Observations by polarized-light optical microscopy also contribute towards identifying paracrystalline phases, in particular when the number of lines observed by x-ray diffraction is insufficient to unambiguously establish the nature of the paracrystalline phases present.

The polarized-light optical microscopy observations are carried out, for example, using a Laborlux S (Leitz) microscope equipped with a magnification 10 objective, a system of cross polarizers and a hot plate (Mettler FP80/FP82). The sample is deposited between a microscope slide and a cover glass, covered with a second slide and the assembly is sealed using a Parafilm® seal. The observations are carried out at various fixed temperatures or by temperature scanning at 2° C./min between ambient temperature and about 95° C.

It is known, for example, that isotropic micellar solutions are non-birefringent, that paracrystalline phases of cubic type are also non-birefringent and that paracrystalline phases of the fluid lamellar type, direct hexagonal or inverse, show various characteristic textures under polarized light, described, for example, in “Textures des phases paracristallines recontrees dans les diagrammes d'equilibre: agents de surface, lipides, eau” [Textures of the paracrystalline phases encountered in equilibrium diagrams: surface agents, lipids, water], F. Lachampt and R. M. Vila, Revue Francaise des corps gras [French review of fatty substances], vol. 2, pp. 87-111 (1969), or in The aqueous phase behavior of surfactants, Robert G. Laughlin, Academic Press, NY, pp. 521-546 (1996), which are incorporated herein by reference.

Surfactant System:

The surfactant system which is used in the composition of the present invention and which makes it possible to obtain a paracrystalline phase during heating to at least 30° C. preferably comprises at least one water-soluble surfactant and at least one water-insoluble surfactant.

The term “water-soluble surfactant” means a surfactant which, at a concentration of 20 g/l in deionized water at a temperature of about 25° C., gives a transparent isotropic solution.

Conversely, the expression “water-insoluble surfactant” means a surfactant which, at a concentration of 20 g/l in deionized water at a temperature of about 25° C., gives a cloudy solution indicating the non-dissolution of the surfactant in the water.

The water-insoluble surfactants form a dispersed phase in the aqueous medium, this dispersed phase comprising all water-insoluble compounds.

The presence of the water-insoluble surfactants allows to improve the qualities of the obtained foam and the creaminess of the composition.

Water-soluble Surfactants:

Any water-soluble surfactant may be used. These are preferably foaming surfactants, i.e., surfactants capable of forming a foam in the presence of water. These are mainly anionic, nonionic or amphoteric derivatives containing fatty chains that are short enough for these products to show good solubility at ambient temperature in the aqueous solvent medium of the composition. A water-soluble surfactant or a mixture of such surfactants may be used.

Water-soluble surfactants which may be mentioned, for example, are:

1. Anionic Surfactants:

According to one particular embodiment of the invention, the surfactant system used preferably comprises at least one water-soluble anionic surfactant, and more particularly at least one water-soluble carboxylic acid or carboxylic acid salt, which salt is obtained from the acid and a base. The carboxylic acids which may be used are fatty acids, that is to say acids comprising a linear or branched, saturated or unsaturated alkyl chain containing from 6 to 16 carbon atoms and preferably 10 to 14 carbon atoms. The salts of such fatty acids are soaps. Whether or not a soap is water-soluble depends on both the length of the alkyl chain and the counterion constituting the salt. Salts which may be used, for example, are alkali metal salts, alkaline-earth metal salts, ammonium salts, amino alcohol salts, and amino acid salts, and in particular the sodium, potassium, magnesium, triethanolamine, N-methylglucamine, lysine, and arginine salts. The bases which can be used to produce these salts can, for example, be inorganic bases, such as alkali metal hydroxides (sodium hydroxide and potassium hydroxide), alkaline earth metal hydroxides (magnesium hydroxide) or ammonium hydroxide, or organic bases, such as triethanolamine, N-methylglucamine, lysine, and arginine. The carboxylic acid may in particular be lauric acid or myristic acid.

Water-soluble soaps which may be mentioned, for example, are the potassium salts of C₁₀ to C₁₄ fatty acids, and in particular the potassium salt of lauric acid, the potassium salt of myristic acid and their mixtures. Soap is generally introduced into the composition in the form of a base, on the one hand, and of the fatty acid, on the other hand, the formation of the salt taking place in situ. Thus, when the water-soluble soap is composed of the potassium salt of lauric acid and/or of the potassium salt of myristic acid, the composition can then comprise lauric acid and/or myristic acid with a sufficient amount of potassium hydroxide to form the potassium salts of lauric acid and/or of myristic acid.

As other anionic surfactants which may be used in the composition of the present invention as water-soluble surfactants, mention may be made, for example, of ethoxylated carboxylic acids and salts thereof; sarconsinates and acyl sarcosinates and salts thereof, such as sodium lauroyl sarcosinate; taurates and methyltaurates and salts thereof; isethionates and acyl isethionates, products of the reaction of fatty acids containing from 10 to 22 carbon atoms with isethionic acid, and salts thereof such as sodium isethionate and sodium cocoyl isethionate; sulfosuccinates and salts thereof;, alkyl sulfates and alkyl ether sulfates and salts thereof, in particular sodium lauryl sulfate or triethanolamine lauryl sulfate, and sodium or potassium lauryl ether sulfate; phosphoric acid monoalkyl and dialkyl esters and salts thereof, such as, for example, sodium monolauryl and dilauryl phosphate, potassium monolauryl and dilauryl phosphate, triethanolamine monolauryl and dilauryl phosphate, sodium monomyristyl and dimyristyl phosphate, potassium monomyristyl and dimyristyl phosphate, diethanolamine monomyristyl and dimyristyl phosphate and triethanolamine monomyristyl and dimyristyl phosphate; alkane sulfonates and salts thereof; bile salts such as cholates, deoxycholates, taurocholates, and taurodeoxycholates; lipoamino acids and salts thereof, such as monosodium and disodium acylglutamates; bipolar geminal surfactants, as described in Surfactant Science series, vol. 74, published by Krister Homberg, which is incorporated herein by reference.

2. Amphoteric and Zwitterionic Surfactants:

As amphoteric or zwitterionic surfactants which may be used as water-soluble surfactants, mention may be made, for example, of betaines such as dimethylbetaine, cocobetaine and cocoamidopropylbetaine; sulfobetaines such as cocoamidopropylhydroxysultaine; alkylamphoacetates such as cocoamphodiacetate; and mixtures thereof.

3. Nonionic Surfactants:

As nonionic surfactants which may be used as water-soluble surfactants, mention may be made, for example, of polyol ethers, comprising fatty chains (8 to 30 carbon atoms), such as oxyethylenated sorbitol or glyceryl fatty ethers; polyglyceryl ethers and esters; polyoxyethylenated fatty alcohols which are ethers formed from ethylene oxide units and at least one fatty alcohol chain containing from 10 to 22 carbon atoms, the solubility of which depends on the number of ethylene oxides and on the length of the fatty chain; for example, for a fatty chain containing 12 carbon atoms, the number of ethylene oxides must be greater than 7, and, by way of example of polyoxyethylenated fatty alcohols, mention may be made of lauryl alcohol ethers comprising more than 7 oxyethylene groups; alkyl polyglucosides in which the alkyl group contains from 1 to 14 carbon atoms (alkyl-C₁-C₁₄ polyglucosides), such as, for example, decylglucoside, laurylglucoside or cocoylglucoside; alkylglucopyranosides and alkylthioglucopyranosides; alkyl maltosides; alkyl-N-methylglucamides; polyoxyethylenated sorbitan esters which generally comprise from 1 to 100 ethylene glycol units and preferably from 2 to 40 ethylene oxide (EO) units; amino alcohol esters; and mixtures thereof.

The surfactant system used in the composition of the present invention comprises a water-soluble surfactant content which may range, for example, from 10% to 50% by weight (of active material), preferably from 15% to 35% by weight, based on the total weight of the composition. According to one preferred embodiment of the present invention, the surfactant system in the composition of the present invention comprises at least 10% by weight, preferably at least 15% by weight, more preferably at least 20% by weight, of a water-soluble surfactant(s) based on the total weight of the composition.

2. Water-insoluble Surfactants:

The water-insoluble surfactants in particular give the texture (consistency) of the final composition. Moreover, in the temperature range between about 25° C. and 45° C., these surfactants partly combine with the water-soluble surfactants to contribute towards the formation of the paracrystalline phase (preferably direct hexagonal) which is the source of the stability of the product up to at least 45° C.

As water-insoluble surfactants used in the composition according to the present invention, mention may be made in particular of water-insoluble carboxylic acids and salts thereof, which salts are obtained from the acid and a base, and thus water-insoluble soaps, that is to say carboxylic acid salts, comprising a linear or branched, saturated or unsaturated alkyl chain containing from 6 to 30 carbon atoms and preferably from 12 to 22 carbon atoms. For the derivatives comprising only one saturated fatty chain, the chain advantageously comprises from 12 to 32 carbon atoms, preferably from 14 to 22 carbon atoms, and better still from 16 to 20 carbon atoms. For the derivatives comprising a monounsaturated or polyunsaturated or branched fatty chain, the chain advantageously comprises from 16 to 34 carbon atoms and preferably from 18 to 24 carbon atoms.

Palmitic acid and stearic acid may be mentioned in particular as the carboxylic acid.

Salts which may be used are alkali metal salts, alkaline-earth metal salts, ammonium salts, amino alcohol salts and amino acid salts, and in particular the sodium, potassium, magnesium, triethanolamine, N-methylglucamine, lysine, and arginine salts. The bases which can be used to produce these salts can, for example, be inorganic bases, such as alkali metal hydroxides (sodium hydroxide and potassium hydroxide), alkaline earth metal hydroxides (magnesium hydroxide) or ammonium hydroxide, or organic bases, such as triethanolamine, N-methylglucamine, lysine, and arginine.

Examples of insoluble soaps which may be mentioned are the sodium salts of C₁₂ to C₂₂ fatty acids and the potassium salts of C₁₆ to C₂₂ fatty acids, and in particular the potassium salt of palmitic acid and the potassium salt of stearic acid.

The soap is generally introduced into the composition in the form of the base, on the one hand, and of the fatty acid, on the other hand, for formation of the salt taking place in situ. Thus, when the insoluble soap is composed of the potassium salt of palmitic acid and/or of the potassium salt of stearic acid, the composition can then comprise palmitic acid and/or stearic acid with a sufficient amount of potassium hydroxide to form the potassium salts of palmitic acid and/or of stearic acid.

As other surfactants which may be used in the composition of the present invention as water-insoluble surfactants, mention may be made, for example, of nonionic or anionic water-insoluble surfactants, for instance glyceryl esters and fatty acid esters containing from 14 to 30 carbon atoms, such as glyceryl stearate; alkyl polyglucosides, the alkyl group for which comprises from 15 to 30 carbon atoms (alkyl-C₁₅-C₃₀ polyglucosides), such as, for example, cetostearyl glucoside; optionally oxyethylenated sterol and phytosterol derivatives; alkali metal salts of cholesteryl sulfate, and in particular the sodium salt; alkali metal salts of cholesteryl phosphate, and in particular the sodium salt; polyoxyethylenated fatty alcohols comprising an oxyethylenated chain with a small number of oxyethylene groups, and in particular less than 10 oxyethylene groups; dialkyl phosphates such as alkali metal salts of dicetyl phosphate, and in particular the sodium and potassium salts; alkali metal salts of dimyristyl phosphate, and in particular the sodium and potassium salts; lecithins; sphingomyelins; ceramides; and mixtures thereof.

The surfactant system used in the composition of the present invention preferably comprises a water-insoluble surfactant content ranging from 5% to 50% by weight (of active material), preferably from 5% to 30% by weight, based on the total weight of the composition.

The surfactant system (water-soluble and water-insoluble surfactants) is present in the composition of the present invention in an amount, of active material, which may range, for example, from 20% to 65% by weight, preferably ranges from 30% to 65% by weight, and better still from 40% to 60% by weight, based on the total weight of the composition. Preferably, the surfactant system comprises an amount of water-soluble soap(s) of at least 10% by weight, relative to the total weight of the composition, and a total amount of soaps (water-soluble and water-insoluble) preferably of at least 20% by weight, relative to the total weight of the composition, and preferably ranging from 30% to 40% by weight, based on the total weight of the composition.

The active agent used in the composition of the invention is chosen from antiseborrhoeic active agents, antimicrobial agents and mixtures thereof. Antiseborrhoeic active agents which may be mentioned, for example, are oestrogens, cyproterone and its acetate, retinoids and aroretinoids (13-cis-retinoic acid), retinol and its derivatives, sulfur and sulfur-containing derivatives, benzoyl peroxide, zinc derivatives such as zinc sulfate, aluminium chloride, selenium disulfide and B vitamins, and mixtures thereof.

The antimicrobial agents may be chosen from antibiotics and antifungal agents, and mixtures thereof. The following active agents may be mentioned, for example, as antimicrobial agents: β-lactam derivatives, quinolone derivatives, ciprofloxacin, norfloxacin, tetracycline and its salts (hydrochloride), erythromycin and its salts (zinc, estolate or stearate salt), amikacin and its salts (sulfate), 2,4,4′-trichloro-2′-hydroxydiphenyl ether (triclosan), 3,4,4′-trichlorobanilide (triclocarban), phenoxyethanol, phenoxypropanol, phenoxyisopropanol, doxycycline and its salts (hydrochloride), capreomycin and its salts (sulfate), chlorhexidine and its salts (gluconate, hydrochloride), chlorotetracycline and its salts (hydrochloride), oxytetracycline and its salts (hydrochloride), clindamycin and its salts (hydrochloride), ethambutol and its salts (hydrochloride), hexamidine and its salts (isethionate), metronidazole and its salts (hydrochloride), pentamidine and its salts (hydrochloride), gentamycin and its salts (sulfate), kanamycin and its salts (sulfate), lineomycin and its salts (hydrochloride), methacycline and its salts (hydrochloride), methenamine and its salts (hippurate, mandelate), minocycline and its salts (hydrochloride), neomycin and its salts (sulfate), netilmicin and its salts (sulfate), paromomycin and its salts (sulfate), streptomycin and its salts (sulfate), tobramycin and its salts (sulfate), miconazole and its salts (hydrochloride), amantadine and its salts (sulfate, hydrochloride), octopirox, para-chloro-meta-xylenol, nystatin, tolnaftate, zinc pyrithione, clotrimazole, salicylic acid, 5-n-octanoylsalicylic acid (or capryloylsalicylic acid), benzoyl peroxide, 3-hydroxybenzoic acid, glycolic acid, lactic acid, 4-hydroxybenzoic acid, acetylsalicylic acid, 2-hydroxybutanoic acid, 2-hydrozypentanoic acid, 2-hydroxyhexanoic acid, phytic acid, N-acetyl-L-cysteine acid, lipoic acid, azelaic acid, arachidonic acid, ibuprofen, naproxen, hydrocortisone, acetaminophen, resorcinol, lidocaine hydrochloride, neomycin sulfate, octoxyglycerol, octanoylglycine (or capryloylglycine), caprylylglycol (1,2-octanediol) and 10-hydroxy-2-decanoic acid, and mixtures thereof.

Some of these active agents may have several functions. For example, salicylic acid may also promote the desquamation of epidermal cells, and may contribute towards normalizing the spreading of sebum on the skin.

In addition to its antibacterial activity on acne-prone skin, azelaic acid inhibits the proliferation of keratinocytes, reduces the content of free fatty acids in sebaceous secretions and has anti-inflammatory activity.

According to one preferred embodiment of the present invention, the active agents used are salicylic acid and its salts, 5-n-octanoylsalicylic acid, benzoyl peroxide, triclosan, phenoxyethanol, octoxyglycerol, octanoylglycine, 10-hydroxy-2-decanoic acid, caprylylglycol, azelaic acid, retinol, and B vitamins and in particular niacinamide. These active agents may be used alone or in combination.

The amount of active agent(s) in the composition according to the present invention depends on the active agent(s) used and may vary within a wide range. It may range, for example, from 0.05% to 20% by weight, preferably from 0.1% to 10% by weight, based on the total weight of the composition.

The composition may also contain one or more anti-inflammatory agents and/or anti-irritant agents such as ursolic acid and oleanolic acid and salts thereof (sold by the company Boehringer under the name “Ursolic oleanic acid”), extract of Paeonia suffruticosa root (sold by Ichimaru Pharcos under the name “Botampi extract powder”), beauty-leaf oil, 18-β-glycyrrhetinic acid and its zinc salt, tamanu oil, Laminaria saccharina extract (sold by Secma under the name “phlorogine”). These active agents may be present in an amount ranging from 0.01% to 10% by weight, based on the total weight of the composition.

The aqueous medium of the foaming creams of the present invention may contain, in addition to water, one or more solvents chosen from lower alcohols containing from 1 to 6 carbon atoms, such as ethanol; polyols such as glycerol; glycols, for instance butylene glycol, isoprene glycol, propylene glycol and polyethylene glycols such as PEG-8; sorbitol; sugars such as glucose, fructose, maltose, lactose or sucrose; and mixtures thereof. The amount of solvent(s) in the composition of the invention may range from 0.5% to 30% by weight, preferably from 5% to 20% by weight, based on the total weight of the composition.

To obtain more or less fluid compositions, one or more thickeners, in particular polymers, may be incorporated into the compositions of the present invention, in preferential concentrations ranging from 0.05% to 2% by weight, based on the total weight of the composition.

Examples of Thickeners Which may be Mentioned are:

polysaccharide biopolymers, for instance xanthan gum, guar gum, alginates, and modified celluloses;

synthetic polymers such as polyacrylics, for instance Carbopol 980 sold by the company Goodrich, acrylate/acrylonitrile copolymers such as Hypan SS201 sold by the company Kingston;

mineral thickeners such as modified or unmodified smectites and hectorites, for instance the Bentone products sold by the company Rheox, the Laponite products sold by the company Southern Clay Products, and the product Veegum HS sold by the company R.T. Vanderbilt; and

mixtures thereof.

The compositions according to the present invention exist as more or less fluid creams which have modulus |G*| values, at a temperature of 25° C., ranging from 10² to 10⁵ Pa and loss angles δ values which range from 10 to 45° for frequencies ranging from 10⁻² to 10 Hz.

|G*| and δ are viscoelastic parameters used to measure the physical properties of viscoelastic fluids, as explained in An introduction to rheology, by H. A. Barnes, J. F. Hutton and K. Walters, Elsevier, NY, pages 46 to 54, 1989, which is incorporated herein by reference.

|G*|i s the modulus of the complex modulus G* and δ is the loss angle. G′ and G″ are the components of G*: G*=G′+iG″. G′ and G″ are, respectively, the storage modulus and the loss modulus, and i is equal to (−1)^{½}. The components G′ and G″ of the complex modulus are obtained from the relationship between the oscillatory stress and the oscillatory strain.

The rheological measurements of |G*| and δ are generally carried out using a Haake RS150 rheometer, at a temperature of 25° C., with cone-plate geometry-measuring bodies, the diameter of the cone and the dimension of the plate being 60 mm and the cone angle 2°, and the gap between the cone and the plate being 0.1 mm.

To carry out dynamic viscoelasticity measurements (oscillatory measurements), the linear viscoelastic domain is first determined, by subjecting the sample to sinusoidal stresses of increasing amplitude and of constant frequency. The modulus values are given as a function of the amplitude of stress or of the amplitude of strain in order to determine the limits of the linear viscoelastic domain. After having identified the linear viscoelastic domain, dynamic measurements are carried out in the linear viscoelastic zone, for a constant strain value in the linear viscoelastic domain and at a variable frequency. The Haake RS150 rheometer can cover a range of frequencies from 0.01 to 10 Hz (i.e., 0.063 to 62.8 rad/sec).

From the values for the amplitudes of stress τ₀, the amplitude of strain γ₀ and the dephasing δ, the following relationships are established:

|G*|=τ₀/γ₀

G′=|G*|cos δ

G″=|G*|sin δ

G*=G′+iG″

The compositions of the present invention may also contain adjuvants commonly used in foaming cleansing agents, for instance cationic polymers of the polyquaternium type, which give the foaming cream softness and smoothness. These cationic polymers may preferably be chosen from the following polymers:

Polyquaternium 5, such as the product Merquat 5 sold by the company Calgon;

Polyquaternium 6, such as the product Salcare SC 30 sold by the company Ciba, and the product Merquat 100 sold by the company Calgon;

Polyquaternium 7, such as the products Merquat S, Merquat 2200 and Merquat 550 sold by the company Calgon, and the product Salcare SC 10 sold by the company Ciba;

Polyquaternium 10, such as the product Polymer JR400 sold by the company Amerchol;

Polyquaternium 11, such as the products Gafquat 755, Gafquat 755N and Gafquat 734 sold by the company ISP;

Polyquaternium 15, such as the product Rohagit KF 720 F sold by the company Rohm;

Polyquaternium 16, such as the products Luviquat FC905, Luviquat FC370, Luviquat HM552 and Luviquat FC550 sold by the company BASF;

Polyquaternium 22, such as the product Merquat 280 sold by the company Calgon;

Polyquaternium 28, such as the product Styleze CC10 sold by the company ISP;

Polyquaternium 39, such as the product Merquat Plus 3300 sold by the company Calgon;

Polyquatemium 44, such as the product Luviquat Care sold by the company BASF;

Polyquaternium 46, such as the product Luviquat Hold sold by the company BASF; and

Polyquaternium 47, such as the product Merquat 2001 sold by the company Calgon.

Cationic guars such as the product Jaguar sold by the company Rhodia may also be used as cationic polymer.

In addition, the compositions of the present invention may contain adjuvants usually used in cosmetics or dermatology, chosen from oils, active agents other than those above-mentioned, fragrances, preserving agents, sequestering agents (EDTA), pigments, nacres, mineral or organic fillers such as talc, kaolin, silica powder or polyethylene powder, soluble colorants and sunscreens. The amounts of these various adjuvants are those used conventionally in the field under consideration, and, for example range from 0.01% to 20% by weight of the total weight of the composition. These adjuvants and their concentrations should be such that they do not modify the property desired for the composition of the invention.

As oils, may be used for example oils of plant origin (jojoba oil, avocado oil, sesame oil, sunflower oil, corn oil, soybean oil, safflower oil, or grape pip oil); mineral oils (petroleum jelly, optionally hydrogenated isoparaffins); synthetic oils (isopropyl myristate, cetearyl octanoate, polyisobutylene, ethylhexyl palmitate or alkyl benzoates); volatile or non-volatile silicone oils such as polydimethylsiloxanes (PDMSs) and cyclodimethylsiloxanes or cyclomethicones; fluoro oils or fluorosilicone oils; as well as mixtures of these oils. The amount of oils must not modify the property desired for the composition of the present invention; it is at most 15% of the total weight of the composition, preferably at most 10% of the total weight of the composition, and it is preferably from 0.1 to 5% of the total weight of the composition and better from 0.1 to 3% of the total weight of the composition.

Active agents which may be mentioned include for example moisturizers and, for example, protein hydrolysates and polyols such as glycerol, glycols, for instance polyethylene glycols, and sugar derivatives; natural extracts; procyannidol oligomers; vitamins; urea; caffeine; depigmenting agents such as kojic acid and caffeic acid; α-hydroxy acids such as lactic acid and glycolic acid; retinoids; screening agents; extracts of algae, of fungi, of plants, of yeasts or of bacteria; hydrolysed, partially hydrolysed or non hydrolysed proteins, enzymes, co-enzyme Q10 or ubiquinone, hormones, vitamins and their derivatives, flavonoides and isoflavones, and mixtures thereof.

The compositions according to the present invention may especially constitute foaming creams for topical application, used in particular in cosmetics or dermatology, for instance cleansing products or makeup-removing products for the skin (body or face, including the eyes), the scalp and/or the hair. A composition for topical use contains a physiologically acceptable medium, that is to say a medium which is compatible with the skin, mucous membranes, the scalp, the eyes and/or the hair. It may more particularly constitute a composition for cleansing greasy skin and/or acne-prone skin. It may also be used for cleansing acne-prone skin before a possible subsequent treatment.

In another embodiment, the present invention provides the cosmetic use of the composition as defined above, as cleansing products and/or make-up-removing products for greasy skin and/or acne-prone skin.

In another embodiment, the present invention provides the use of a composition as defined above to prepare a composition for cleansing acne-prone skin.

In another embodiment, the present invention provides a cosmetic process for cleansing greasy skin and/or acne-prone skin, characterized in that the composition of the invention is applied to the skin, in the presence of water, in that it is massaged to form a foam and in that the foam formed is removed by rinsing with water.

In a preferred embodiment, the present compositions are used to cleanse greasy or acne-prone skin, typically the skin of the face and back and even more preferably the skin of the face. In this method, the composition of the present invention is mixed with water to form a foam which is used to cleanse the skin. Typically the foam is formed by mixing about 0.3 to about 5 g, preferably about 0.5 to about 2 g, even more preferably about 1 g of the composition with about 0.5 to about 30 g, preferably about 1 to about 20 g, even more preferably about 2 to 6 g of water. The mixing of the composition with the water to form the foam may be carried out by massaging the composition with the water in the palms of the user and the resulting foam applied to the face or back. Alternatively, the foam may be formed by massaging the composition directly onto the skin of the face or back which has already been wetted. Once the foam has been applied to or formed on the skin, the foam is then massaged on the skin to effect cleansing for a period of a few minutes. After the cleansing has been completed, the foam is removed from the skin by rinsing with water. The process may be repeated if necessary or desired. The present compositions may be used in a cleansing regime which involves cleansing with the present composition monthly, weekly, every other day, daily, or twice a day (such as in the morning and evening). The use of the present composition for cleansing the skin may be carried out for a time sufficient to achieve the desired reduction in greasiness of the skin or the desired reduction in acne. Alternatively, the cleansing of the skin with the present composition may be continued for an indefinite period of time for preventative purposes.

Other features of the invention will become apparent in the course of the following descriptions of exemplary embodiments which are given for illustration of the invention and are not intended to be limiting thereof.

### EXAMPLES

In the following examples, and throughout this specification, all parts and percentages are by weight based on the total weight of the composition, and all temperatures are in degrees Celsius, unless expressly stated to be otherwise.

### Example According to the Invention

### Foaming Cream for Cleansing Greasy Skin

| | |
|---|---|
| Preserving agent | 0.4% |
| Tetrasodium EDTA | 0.2% |
| Potassium hydroxide | 7% |
| Glycerol | 7% |
| PEG-8 | 7% |
| Lauric acid | 3% |
| Myristic acid | 20% |
| Palmitic acid | 3% |
| Stearic acid | 3% |
| Glyceryl stearate (INCI name: Glyceryl | 5% |
| stearate SE) | |
| Cocoylglucoside (at 50% active material) | 2% |
| (i.e. 1% active material) | |
| 5-n-Octanoylsalicylic acid (Mexoryl SAM sold | 0.3% |
| by the company Chimex) | |
| Water | qs 100% |

Procedure:

The aqueous phase consisting of the water-soluble ingredients (water, preserving agents, EDTA, glycerol, PEG-8) is brought to 80° C. The fatty phase consisting of the fatty acids, the glyceryl stearate and the 5-n-octanoylsalicylic acid is heated to 80° C. and added with stirring to the aqueous phase. The cocoylglucoside is then added, followed by the potassium hydroxide dissolved in some of the water. Stirring is continued for 10 minutes at 80° C. and the mixture is then cooled with stirring.

The Obtained Foaming Composition is Creamy and Soft.

The water-soluble surfactants consisting of the potassium salts of lauric and myristic acids, and the cocoylglucoside, represent 29.5% by weight of the composition, while the water-insoluble surfactants, consisting of the potassium salts of palmitic and stearic acids, and the glyceryl stearate, represent 12.5% by weight of the composition. The composition thus contains 42% by weight of surfactants in total, in which 36% by weight are soaps (KOH+lauric, myristic, palmitic, and stearic acids). The water-soluble soaps represent 28.5% by weight of the weight of the composition.

The composition obtained has the appearance of a white cream at room temperature; it is converted into an extremely viscous translucent gel at between 35 and 40° C.; this gel exists up to 75-80° C., at which temperature the composition becomes fluid. On returning to an ambient temperature of 25° C., this gel regains the appearance of homogeneous cream.

This cream is entirely stable at 4° C., at ambient temperature and at 45° C. for at least two months.

For this cream, the |G*| values are 2,900 Pa at 0.01 Hz and 25,000 Pa at 1 Hz, and the values of δ are 45° at 0.01 Hz and 40° at 1 Hz.

Characterization:

At 25° C., the cream consists of a micellar phase which was able to be isolated by centrifugation (for one hour at 64,000× g, i.e., at 30,000 rpm, with a Sigma 3K30 centrifuge equipped with a 1210 rotor) and a crystalline phase. The micellar phase is transparent, fluid and nonbirefringent in polarized light, in x-ray diffraction. This phase gives a broad line at small angles which is centred around a distance d=49.8 Å and a band at large angles which is centred about a distance d=4.64 Å.

On the basis of the measurements carried out on the whole cream, the crystalline phase has a melting point: Tm=42° C. by DSC and is characterized on x-ray diffraction by 3 fine lines at small angles corresponding to distances d=42.7; 21.4; and 14.2 Å and 7 fine lines at large angles corresponding to d=4.37; 4.27; 4.19; 3.92; 3.68; 3.35; and 3.07 Å.

At 35° C., the cream is homogeneous at the macroscopic scale and consists of a mixture of a hexagonal phase characterized on x-ray diffraction by 2 fine lines at small angles corresponding to distances d=48.7 and 24.3 Å, and a crystalline phase characterized by 3 fine lines at small angles corresponding to d=42.0; 21.0; and 14.0 Å and 3 fine lines at large angles corresponding to d=4.29; 3.92; and 3.09 Å.

At 45° C., the cream is homogeneous at the macroscopic scale and consists of a mixture containing a hexagonal phase, characterized on x-ray diffraction by 2 fine lines at small angles corresponding to distances d=49.6 and 28.7 Å, and a fluid lamellar phase characterized by 1 fine line at small angles corresponding to d=45.0 Å. At large angles, a band centred about 4.60 Å is observed, in accordance with the presence of paracrystalline phases.

At 55° C., the cream is homogeneous at the macroscopic scale and consists of a mixture containing a hexagonal phase, characterized on x-ray diffraction by 2 fine lines at small angles corresponding to distances d=47.7 and 27.5 Å, and a fluid lamellar phase characterized by 1 weak fine line at small angles corresponding to d=36.5 Å. At large angles, a band centred about 4.70 Å is observed, in accordance with the presence of paracrystalline phases.

### Comparative Example

| | |
|---|---|
| Preserving agent | 0.4% |
| Tetrasodium EDTA | 0.2% |
| Potassium hydroxide | 4% |
| Glycerol | 7% |
| PEG-8 | 7% |
| Lauric acid | 3% |
| Myristic acid | 3% |
| Palmitic acid | 8.7% |
| Stearic acid | 8.7% |
| Glyceryl stearate (INCI name: Glyceryl | 0.75% |
| stearate SE) | |
| Sodium lauroyl sarcosinate (containing 30% | 21.8% |
| active material) (i.e. about 7% active material) | |
| 5-n-Octanoylsalicylic acid (Mexoryl SAB sold | 0.3% |
| by the company Chimex) | |
| Water | qs 100% |

The procedure is the same as in Example 1.

The water-soluble surfactants consisting of the potassium salts of lauric and myristic acid, and the lauroyl sarcosinate, represent 14% by weight of the composition, while the water-insoluble surfactants, consisting of the potassium salts of palmitic and stearic acid, and the glyceryl stearate, represent 21.15% by weight of the composition. The composition thus contains 35.15% by weight of surfactants, of which 27.4% by weight are soaps (KOH+lauric, myristic, palmitic, and stearic acids). The water-soluble soaps represent 7% by weight of the weight of the composition.

The composition obtained has the appearance of a white cream at room temperature. This composition is entirely stable at 4° C., but is unstable at 45° C. and separates into two phases. On returning to ambient temperature, it is heterogeneous.

At 25° C., the cream consists of a micellar phase which was able to be isolated by centrifugation (for one hour at 64,000× g, i.e., at 30,000 rpm, with a Sigma 3K30 centrifuge equipped with a 1210 rotor) and a crystalline phase.

The micellar phase is transparent, fluid and nonbirefringent in polarized light. In x-ray diffraction, this phase gives a broad line at small angles which is centred around a distance d 50.0 Å and a band at large angles which is centred around a distance d=4.53 Å.

On the basis of the measurements carried out on the whole cream, the crystalline phase has a melting point: Tm=45° C. by DSC and is characterized on x-ray diffraction by 5 fine lines at small angles corresponding to distances d=49.0; 24.1; 16.0; 12.2; and 9.64 Å and 6 fine lines at large angles corresponding to d=4.37; 4.28; 4.20; 3.94; 3.66; and 3.08 Å.

At 50° C., macroscopic demixing of the cream into two phases takes place: an upper phase of fluid lamellar type which has, in polarized light, a characteristic texture of the type: “Maltese cross” and gives, on x-ray diffraction, a fine line at small angles corresponding to 43.3 Å and a band at large angles which is centred about 4.78 Å.

A lower phase of micellar solution type, which is fluid, transparent and nonbirefringent in polarized light and characterized on x-ray diffraction by a diffuse line at small angles corresponding to d=58.0 Å and a band at large angles which is centred about 4.80 Å.

The essential difference between the composition of the example according to the present invention and the composition of the comparative example relates to the macroscopic appearance above 45° C.: the composition according to the present invention gives a homogeneous system, while the composition of the comparative example results in demixing.

For the composition according to the present invention of Example 1, the system consists above 45° C. of a lamellar phase mixed with a direct hexagonal phase, the high viscosity of which makes it possible to avoid macroscopic demixing.

For the composition of the comparative example, the system consists above 45° C. of a lamellar phase mixed with a micellar phase, the low viscosity of which does not make it possible to avoid macroscopic demixing, resulting in a heterogeneous composition on returning to ambient temperature.

### Example 2

### Foaming Cream For Cleansing Greasy Skin

| | |
|---|---|
| Preserving agent | 0.4% |
| Tetrasodium EDTA | 0.2% |
| Potassium hydroxide | 7% |
| Glycerol | 7% |
| PEG-8 | 7% |
| Lauric acid | 3% |
| Myristic acid | 20% |
| Palmitic acid | 3% |
| Stearic acid | 3% |
| Glyceryl stearate (INCI name: Glyceryl | 5% |
| stearate SE) | |
| Cocoylglucoside (at 50% active material) | 2% |
| (i.e. 1% active material) | |
| Azelaic acid (Emerox 1144 Azelaic Acid sold | 5% |
| by the company Cognis) | |
| Water | qs 100% |

Procedure:

The aqueous phase consisting of the water-soluble ingredients (water, preserving agents, EDTA, glycerol, PEG-8, azelaic acid) is brought to 80° C. The fatty phase consisting of the fatty acids and the glyceryl stearate is heated and added with stirring to the aqueous phase. The cocoylglucoside is then added, followed by the potassium hydroxide dissolved in some of the water. Stirring is continued for 10 minutes at 80° C., and the mixture is then cooled with stirring.

The same characteristics as in Example 1 are obtained.

Obviously, numerous modifications and variations of the present invention are possible in light of the above teachings. It is therefore to be understood that, within the scope of the appended claims, the invention may be practiced otherwise than as specifically described herein.

All patents and other references mentioned above are incorporated in full herein by this reference, the same as if set forth at length.

1. A foaming composition which is a cream for topical application, and which comprises, in an aqueous medium:
(1) a surfactant system such that at least one paracrystalline phase of direct and/or cubic hexagonal type appears when the temperature increases above 30° C. and such that said paracrystalline phase remains present up to at least 45° C.; and
(2) an active agent selected from the group consisting of anti-seborrhoeic active agents and antimicrobial agents.

2. The composition according to claim 1, wherein said paracrystalline phase comprises at least one direct hexagonal phase.

3. The composition according to claim 1, which has a modulus |G*| ranging, at a temperature of 25° C., from 10² to 10⁵ Pa and a loss angle δ ranging from 10° to 45° for frequencies ranging from 10⁻² to 10 Hz.

4. The composition according to claim 1, wherein said surfactant system comprises at least one water-soluble surfactant and at least one water-insoluble surfactant.

5. The composition according to claim 4, wherein said surfactant system comprises at least one water-soluble anionic surfactant.

6. The composition according to claim 4, wherein said water-soluble anionic surfactant is selected from the group consisting of carboxylic acids and salts thereof, ethoxylated carboxylic acids and salts thereof, sarcosinates and acyl sarcosinates and salts thereof, taurates and methyltaurates and salts thereof, isethionates and acyl isethionates and salts thereof, sulfosuccinates and salts thereof, alkyl sulfates and alkyl ether sulfates and salts thereof, monoalkyl and dialkyl esters of phosphoric acid and salts thereof, alkane sulfonates and salts thereof, bile salts, lipoamino acids and salts thereof, geminal surfactants, and mixtures thereof.

7. The composition according to claim 4, wherein said water-soluble surfactant is an amphoteric or zwitterionic surfactant selected from the group consisting of betaines, sulfobetaines and alkylamphoacetates, and mixtures thereof.

8. The composition according to claim 4, wherein said water-soluble surfactant is a nonionic surfactant selected from the group consisting of polyol ethers, polyglyceryl ethers and esters, polyoxyethylenated fatty alcohols, alkyl-C₁-C₁₄-polyglucosides, alkyl glucopyranosides and alkylthioglycopyranosides, alkyl maltosides, alkyl-N-methylglucamides, polyoxyethylenated sorbitan esters, amino alcohol esters, and mixtures thereof.

9. The composition according to claim 4, wherein said water-insoluble surfactant is selected from the group consisting of carboxylic acids and salts thereof; glyceryl esters and fatty acid esters; alkyl-C₁₅-C₃₀-polyglucosides; optionally oxyethylenated sterol and phytosterol derivatives; alkali metal salts of cholesteryl sulfate; alkali metal salts of cholesteryl phosphate; polyoxyethylenated fatty alcohols; dialkyl phosphates; lecithins; sphingomyelins; ceramides, and mixtures thereof.

10. The composition according to claim 1, wherein said surfactant system is present in an amount, of active material, ranging from 20% to 65% by weight, relative to the total weight of the composition.

11. The composition according to claim 1, wherein said surfactant system comprises from 10% to 50% by weight of a water-soluble surfactant, relative to the total weight of the composition.

12. The composition according to claim 11, wherein said surfactant system comprises at least 15% by weight of a water-soluble surfactant, relative to the total weight of the composition.

13. The composition according to claim 1, wherein said surfactant system comprises at least 10% by weight of a water-soluble soap, relative to the total weight of the composition.

14. The composition according to claim 1, wherein said surfactant system comprises from 5% to 50% by weight of a water-insoluble surfactant, relative to the total weight of the composition.

15. The composition according to claim 1, wherein said surfactant system comprises a total amount of soap of at least 20% by weight, relative to the total weight of the composition.

16. The composition according to claim 1, wherein said active agent is selected from the group consisting of oestrogens, cyproterone and its acetate, retinoids and aroretinoids, retinol and its derivatives, sulfur and sulfur-containing derivatives, benzoyl peroxide, zinc derivatives, aluminium chloride, selenium disulfide, B vitamins, P-lactam derivatives, quinolone derivatives, ciprofloxacin, norfloxacin, tetracycline and its salts, erythromycin and its salts, amikacin and its salts, triclosan, triclocarban, phenoxyethanol, phenoxypropanol, phenoxyisopropanol, doxycycline and its salts, capreomycin and its salts, chlorhexidine and its salts, chlorotetracycline and its salts, oxytetracycline and its salts, clindamycin and its salts, ethambutol and its salts, hexamidine and its salts, metronidazole and its salts, pentamidine and its salts, gentamycin and its salts, kanamycin and its salts, lineomycin and its salts, methacycline and its salts, methenamine and its salts, minocycline and its salts, neomycin and its salts, netilmicin and its salts, paromomycin and its salts, streptomycin and its salts, tobramycin and its salts, miconazole and its salts, amantadine and its salts, octopirox, para-chloro-meta-xylenol, nystatin, tolnaftate, zinc pyrithione, clotrimazole, salicylic acid, 5-n-octanoylsalicylic acid, 3-hydroxybenzoic acid, glycolic acid, lactic acid, 4-hydroxybenzoic acid, acetylsalicylic acid, 2-hydroxybutanoic acid, 2-hydroxypentanoic acid, 2-hydroxyhexanoic acid, phytic acid, N-acetyl-L-cysteine acid, lipoic acid, azelaic acid, arachidonic acid, ibuprofen, naproxen, hydrocortisone, acetaminophen, resorcinol, lidocaine hydrochloride, neomycin sulfate, octoxyglycerol, octanoylglycine, caprylylglycol, 10-hydroxy-2-decanoic acid, and mixtures thereof.

17. The composition according to claim 1, wherein the amount of said active agent ranges from 0.05% to 20% by weight, relative to the total weight of the composition.

18. The composition according to claim 1, which further comprises at least one or more anti-inflammatory and/or anti-irritant agents.

19. The composition according to claim 1, which further comprises at least one solvent selected from the group consisting of lower alcohols, polyols, sugars, and mixtures thereof.

20. The composition according to claim 1, which further comprises at least one thickener.

21. The composition according to claim 1, which is a cleansing composition for greasy skin and/or acne-prone skin.

22. A method for cleansing greasy skin and/or acne-prone skin, said method comprising:
(A) applying to the skin of a subject in need thereof, an effective amount of a composition in the presence of water;
(B) massaging said composition to form a foam; and
(C) removing said foam by rinsing with water, wherein said composition comprises, in an aqueous medium:
(1) a surfactant system such that at least one paracrystalline phase of direct and/or cubic hexagonal type appears when the temperature increases above 30° C. and such that this paracrystalline phase remains present up to at least 45° C.; and
(2) an active agent chosen from anti-seborrhoeic active agents and antimicrobial agents.
